(19) United States
(12) Patent Application Publication
(10) Pub. No.: US 2010/0261965 A1
(43) Pub. Date: Oct. 14, 2010

(54) Apparatus For Fluorescence Diagnosis

(76) Inventors: Gerd Beck, Wurmlingen (DE); Andre Ehrhardt, Wurmlingen (DE); Klaus-Martin Irion, Emmingen-Liptingen (DE)

Correspondence Address: ST. ONGE STEWARD JOHNSTON & REENS, LLC, 986 BEDFORD STREET, STAMFORD, CT, 06905-5619, US

(21) Appl. No.: 12/755,144

(22) Filed: Apr. 6, 2010

(30) Foreign Application Priority Data
Apr. 8, 2009 (DE) 10 2009 018 141.5

(51) Int. Cl. A61B 1/06; G01J 1/58

(52) U.S. Cl. 600/160; 250/458.1

(57) ABSTRACT

An apparatus for fluorescence diagnosis comprises an illumination system for illuminating a target area. The illumination system has a light source designed for generating white light and an illumination path. The apparatus further comprises an observation system for observing the target area, which has an observation path. The illumination system has a first illumination mode, in which an illumination spectral filter is arranged in the illumination path, which filter is transmitting in an excitation spectral range and substantially non-transmitting in the spectral range on the long-wavelength side outside of the excitation spectral range, and said illumination system has a second illumination mode, in which the illumination spectral filter is not arranged in the illumination path. An observation spectral filter is arranged in the observation path in the first illumination mode and in the second illumination mode, which filter is substantially non-transmitting in the excitation spectral range and transmitting in the spectral range on the long-wavelength side outside of the excitation spectral range. The observation spectral filter additionally is transmitting in the spectral range on the short-wavelength side outside of the excitation spectral range.

### CROSS-REFERENCE TO FOREIGN APPLICATION

The present application claims priority of German patent application No. 10 2009 018 141.5 filed on Apr. 8, 2009.

### BACKGROUND OF THE INVENTION

The invention generally relates to apparatuses for fluorescence diagnosis. More specifically, the invention relates to an apparatus for fluorescence diagnosis, comprising an illumination system for illuminating a target area, which illumination system has a light source designed for generating white light, and an observation system for observing said target area. The apparatus further comprises an illumination spectral filter and an observation spectral filter.

An apparatus according to the invention for fluorescence diagnosis of the type mentioned at the outset can be used for medical diagnostic purposes, but also for technical diagnostic purposes in industrial or scientific applications. Although hereafter the invention will be described with reference to the medical fluorescence diagnosis, the invention is not limited thereto.

An apparatus known from the document EP 0 861 044 B1 is used for medical fluorescence diagnosis.

The medical fluorescence diagnosis is used for evaluating the state of biological tissue, for example for detecting a tumour, but also for detecting perfusion and vitality. An apparatus for fluorescence diagnosis of the type mentioned at the outset in particular can perform the fluorescence diagnosis in vivo.

In terms of the present invention, a “fluorescent substance” can be a fluorescent dye, a pigment, etc., which was previously introduced into the target area, for example by dispensing the fluorescent substance or a precursor thereof to a patient (human or animal). However, a “fluorescent substance” can also be a substance already available in the target area, for example a tissue-specific substance in the case of medical fluorescence diagnosis, which is excited to autofluoresce by the fluorescence diagnosis. The present invention can comprise both cases. In the following explanations, the assumption is made that the fluorescent substance is a fluorescent dye, which is artificially introduced into the target area, i.e. the tissue region to be examined.

For this purpose, the fluorescent dye or an initial state of this fluorescent dye is dispensed to the patient to be examined. In fluorescence diagnosis for tissue examination carried out by the known apparatus, 5-aminolevulinic acid is dispensed to the patient as an initial substance, from which the fluorescent dye protoporphyrin IX is formed intracellularly by heme biosynthesis. In fluorescence diagnosis, the fact that the fluorescent dye, in the above case protoporphyrin IX, accumulates more strongly in malignant tissue, for example tumour tissue, than in healthy tissue is utilized.

The fluorescent dye can be excited to fluoresce by illumination with light in a spectral range absorbed by the fluorescent dye. The fluorescent dye protoporphyrin IX absorbs light most strongly in a spectral range around 400 nm (Soret-band), that is to say in the violet-blue visible spectral range. The fluorescent light is always of a longer wavelength than the excitation light, and, in the case of the fluorescent dye protoporphyrin, the fluorescence emission is in the visible spectral range with a maximum at 635 nm and a further peak at 705 nm, that is to say in the red visible spectral range. The intensity of the fluorescent light is significantly lower than the intensity of the excitation light.

The known apparatus for fluorescence diagnosis has an illumination system for illuminating the target area, i.e. a tissue region, that is intended to be examined for the presence of malignant tissue and in which the fluorescent dye is present, and an observation system for observing the tissue region. The illumination system has a light source designed to generate white light.

The white light is directed into the tissue region along the illumination path in order to excite the fluorescent dye present therein to fluoresce. Since the white light contains, more or less continuously, the entire wavelength spectrum of at least the visible spectral range, but, by contrast, it is only the fluorescent dye that is intended to be excited to fluoresce, an illumination spectral filter is arranged in the illumination path of the illumination system, which filter is transmitting (transparent) in an excitation spectral range which at least partly lies in the absorption spectral range of the fluorescent dye, and which filter is substantially non-transmitting (opaque) in the spectral range on the long-wavelength side outside of the excitation spectral range. This prevents illumination light reflected from the tissue region from outshining the significantly weaker fluorescence, which, as mentioned above, lies in the spectral range on the long-wavelength side outside of the absorption spectral range. In the observation path, there is arranged an observation spectral filter, which, by contrast, is substantially non-transmitting (opaque) in the excitation spectral range and transmitting (transparent) in the spectral range on the long-wavelength side outside of the excitation spectral range. Thus, the observation filter is substantially non-transmitting to the excitation light in the excitation spectral range reflected in the tissue region and, by contrast, allows the fluorescent light to pass to be observed by the eye, an observation instrument such as an endoscope, a microscope or a camera.

If the observation spectral filter were to be completely non-transmitting to the excitation light passed by the illumination spectral filter, the observation system would only be able to observe the fluorescence originating from the fluorescent dye (and possibly unspecific fluorescence originating from the tissue, see below), which fluorescence is spatially restricted to those regions in the target area in which the fluorescent dye is present. In the other regions, in which there is no fluorescent dye, no light can be observed through the observation system, which makes orientation in the observed target area difficult to impossible. Moreover, it is impossible in practice to distinguish completely between the fluorescence from the fluorescent dye and the fluorescence of endogenous fluorochromes, as a result of which healthy tissue is displayed in dark red in fluorescence images recorded in this fashion and malignant tissue regions are correspondingly brighter. However, it is often difficult to distinguish between healthy and malignant tissues in such images.

It is for this reason that the observation spectral filter is matched to the illumination spectral filter such that the observation spectral filter lets just so much excitation light pass that it outshines, compared to the specific fluorescence to be observed, the even weaker unspecific fluorescence and that the tissue regions in which there is no fluorescent dye can also be observed with sufficient brightness, with these regions appearing blue with respect to the fluorescent dye protoporphyrin IX in the case of the known apparatus for fluorescence diagnosis, while the fluorescence appears red.

The preceding discussions relate to the first mode of operation of the illumination system, which is also referred to as fluorescence mode.

However, the known apparatus for fluorescence diagnosis can also be operated in a second operating mode of the illumination system, in which the illumination spectral filter is removed from the illumination path and so all the white light reaches the target area via the illumination path. This operating mode is used to provide the observer with a natural display of the observed target area. This mode of operation, also referred to as white-light mode, should permit an observation of the target area with a colour reproduction that is as natural as possible. In the known apparatus for fluorescence diagnosis, the observation spectral filter can remain in the observation path in the white-light mode because the observation spectral filter of the known apparatus is transmitting in the visible spectral range above approximately 450 nm. Thus, the observation spectral filter only cuts off the spectral range below approximately 450 nm, and thus it is only a small spectral component in the blue spectral range of the light reflected by the target area that is discarded. The image of the target area obtained in this fashion corresponds almost exactly to the usual white-light image in respect of its colour fidelity, wherein image processing measures such as white-balancing can further improve the natural colour display.

The known apparatus for fluorescence diagnosis can thus be switched between the fluorescence mode and the white-light mode without having to remove the observation spectral filter from the observation path. However, this is made possible in the known apparatus by the “expedient” position of the absorption spectral range of the fluorescent dye protoporphyrin IX.

Other fluorescent dyes used in the medical diagnosis have absorption spectrums that lie in the mid- or long-wavelength region of the visible spectral range, as is the case, for example, in the fluorescent dye indocyanine green and the derivatives thereof, and substances with similar properties. If the concept of the known apparatus were to be transferred to such fluorescent dyes, the short-wavelength end of the pass band of the observation spectral filter would have to be shifted toward longer wavelengths, as a result of which a larger part of the visible spectrum of the light reflected in the target area would not contribute to the lifelike colour display of the observed target area in the white-light mode. The colour impression of the observed target area would then be falsified in an unacceptable fashion. Therefore, the observation spectral filter, present in the observation path in the fluorescence mode, would have to be removed from the observation path in the white-light mode in order to be able to observe a lifelike white-light image. However, this would disadvantageously increase the manufacturing complexity in the design of the observation system, and thus disadvantageously increase the production costs of the apparatus as a result of the provision of a filter changer.

DE 41 33 493 A1 (Asahi K.K.) discloses an apparatus for fluorescence diagnosis, in which there is in the illumination path of the illumination system an illumination spectral filter, which is transmitting in the spectral range between 400 nm and 500 nm. In the observation path of the observation system there is an observation spectral filter, which is transmitting in the spectral range between 500 nm and 600 nm. This known apparatus can only be operated in the fluorescence mode.

Further apparatuses for fluorescence diagnosis are disclosed in U.S. Pat. No. 6,899,675, U.S. Pat. No. 5,590,660 and U.S. Pat. No. 6,061,591.

### SUMMARY OF THE INVENTION

Therefore, the invention is based on the object of developing an apparatus for fluorescence diagnosis such that, independently of the fluorescent substance, both a fluorescence image in the fluorescence mode and a colour display of the target area in the white-light mode, with the colour display being as lifelike as possible, are possible without having to remove the observation spectral filter from the observation path in the white-light mode.

According to the invention, an apparatus for fluorescence diagnosis is provided, comprising
an illumination system for illuminating a target area, the illumination system having
   a light source designed for generating white light,
   an illumination path,
   an illumination spectral filter, the illumination spectral filter being transmitting in an excitation spectral range and substantially non-transmitting in a spectral range on a long-wavelength side outside of the excitation spectral range,
   the illumination spectral filter being arranged in the illumination path in a first illumination mode and being removed from the illumination path in a second illumination mode,
an observation system for observing the target area, the observation system having
   an observation path,
   an observation spectral filter, the observation spectral filter being substantially non-transmitting in the excitation spectral range and transmitting in the spectral range on the long-wavelength side outside of the excitation spectral range, and further being transmitting in a spectral range on a short-wavelength side outside of the excitation spectral range,
   the observation spectral filter being arranged in the observation path in the first illumination mode and in the second illumination mode.

Accordingly, the apparatus for fluorescence diagnosis according to the invention has in the observation path of the observation system an observation spectral filter that has two pass bands, to be precise a first pass band on the long-wavelength side outside of the excitation spectral range and a second pass band on the short-wavelength side outside of the excitation spectral range. In the excitation spectral range itself, the observation spectral filter is substantially non-transmitting, it being understood that the observation spectral filter can still have a small amount of remaining transmission of, for example, less than approximately 5% in the excitation spectral range. If the absorption spectral range of the fluorescent substance now lies completely within the visible spectral range, the apparatus according to the invention has the advantage that, in the white-light mode, the observation spectral filter largely transmits all spectral components of the white light through it, with the exception of the excitation spectral range substantially masked by the observation spectral filter, and so a largely natural colour impression is created when observing the target area in the white-light mode, in which the illumination spectral filter is removed from the illumination path. In the fluorescence mode, in which the illumination spectral filter is arranged in the illumination path, the fluorescence image can be transmitted by the apparatus according to the invention using the same observation spectral filter as is used in the white-light mode. Hence, the observation spectral filter in the apparatus according to the invention can remain in the observation path in both the fluorescence mode and the white-light mode, as a result of which the manufacturing complexity of the apparatus according to the invention is significantly reduced or simplified. By way of example, a changer for changing, removing or inserting the observation spectral filter in the observation system can be dispensed with. This also allows the observation system to switch significantly faster between the fluorescence mode and the white-light mode.

The apparatus according to the invention for fluorescence diagnosis is not limited to a particular fluorescent substance. For the apparatus according to the invention, it holds true that the principle according to the invention also can be transferred to other fluorescent substances. In general, what holds true is that a fluorescent substance considered is absorbent in an absorption spectral range between two spectral boundaries Y1 and Y2, wherein the natural constants Y1 and Y2 are preferably measured in vivo. The spectral boundaries Y1 and Y2 can both, or partly, lie in the visible spectral range. The observation spectral filter in terms of the present invention is transmitting on the short-wavelength side of the spectral boundary Y1 up to a spectral boundary Y1−ΔX, and on the long-wavelength side above a spectral boundary Y2+ΔX. ΔX can in this case lie in the region of approximately 15 nm to approximately 45 nm, wherein ΔX should be as small as possible, depending on the technical implementability of spectral filters.

Moreover, the excitation spectral range, in which the illumination spectral filter is transmitting and which is masked by the observation spectral filter, should be as narrow as possible. Thus, provision is made in a preferred refinement for the excitation spectral range to be approximately as wide as or narrower than an absorption spectral range of a fluorescent substance in the target area.

This advantageously allows the largest possible part of the visible spectral ranges to be used in the white-light mode for a lifelike colour reproduction of the image of the target area.

In a further preferred refinement, the observation spectral filter is transmitting at least up into the near infrared on the long-wavelength side outside of the excitation spectral range and/or said observation spectral filter is transmitting up into the UV spectral range on the short-wavelength side outside of the excitation spectral range.

In this case, it is advantageous that, in the white-light mode, the short-wavelength and/or long-wavelength components of the visible spectral range of the illumination light reflected in the target area also can contribute to colouring that is as lifelike as possible.

In a further preferred refinement, the illumination spectral filter has a first transmission characteristic and the observation spectral filter has a second transmission characteristic, with the first transmission characteristic is complementary to the second transmission characteristic.

The advantage of this measure is that the observation spectral filter can have a high transmission up to the vicinity of the excitation spectral range, to be precise both on the short-wavelength and long-wavelength side of the excitation spectral range, as a result of which, in the white-light mode, even the spectral components of the reflected illumination light up to the vicinity of the excitation spectral range can contribute to colour imaging that is as lifelike as possible. In principle, what holds true is that the observation spectral filter and the illumination spectral filter are optimally matched to the excitation spectral range in respect of their transmission in order, on the one hand, to be able clearly to observe the fluorescence in the fluorescence mode and, on the other hand, to obtain a colour display that is as lifelike as possible in the white-light mode.

In a further preferred refinement, the observation spectral filter has a transmission in a range of between approximately 0.05% and approximately 5% in the excitation spectral range.

In the fluorescence mode, the advantage of this measure is that the colour contrast between the specific fluorescence to be observed and the unspecific fluorescence appearances is increased in the fluorescence image. Just enough of the illumination light in the excitation spectral range is added to the fluorescence image that there is an optimal colour contrast. Moreover, the low-intensity added illumination light improves the orientation of the observer in the fluorescence image. The subjectively perceived visual colour contrast is also increased, which leads to a clearer distinction in the tissue region.

Within the scope of the aforementioned refinement, the observation spectral filter can have a transmission in a range of between approximately 0.05% and approximately 5% in the excitation spectral range, either at points or over the entire spectral band of the excitation spectral range or in one or more subbands of the excitation spectral range.

In a further preferred refinement, the illumination spectral filter has a first transmission function, which at its short-wavelength and/or long-wavelength end has an edge with a gradient between the transmission minimum and the transmission maximum in a range of approximately 100%/(45 nm) to approximately 100%/(10 nm).

Using an illumination spectral filter with a transmission function with a steep edge at its short-wavelength and/or long-wavelength end is advantageous in that the pass band of the observation spectral filter at the long-wavelength end thereof adjacent to the excitation spectral range and/or at the short-wavelength end thereof adjacent to the excitation spectral range can reach very close to the pass band of the illumination spectral filter, which in turn contributes to an improved colour display in the white-light mode, without however the pass bands of the observation spectral filter reaching into the pass hand of the illumination spectral filter, as a result of which the less-intense fluorescence can be observed with a high contrast in the fluorescence mode.

Similarly, it is preferred if the observation spectral filter has a second transmission function, which at its short-wavelength and/or long-wavelength end adjacent to the first transmission function has an edge with a gradient between the transmission minimum and the transmission maximum in a range of approximately 100%/(45 nm) to 100%/(10 nm).

A maximum gradient of the edges of the first transmission function and the second transmission function that can be implemented from a technical point of view is preferred for the present invention. This allows matching of the pass bands of the observation spectral filter and the excitation spectral filter with a minimum spectral distance between the edges and at the same time a high definition between these pass bands.

In a further preferred refinement, a spectral distance between a 50% transmission value of the observation spectral filter and a 50% transmission value of the illumination spectral filter at the short-wavelength and/or long-wavelength end of the excitation spectral range is less than approximately 45 nm, preferably less than approximately 20 nm, and more preferably said spectral distance is approximately 15 nm.

This measure is particularly advantageous when combined with the above-described refinements, according to which the transmission functions of the illumination spectral filter and the observation spectral filter have a steep linear edge. Using such a small spectral distance between the 50% transmission values of the edges of the transmission functions of the observation spectral filter and the illumination spectral filter, this simultaneously achieves a sufficient separation of the pass bands and a small spectral distance between the pass bands.

In preferred refinements, the 50% transmission value of the observation spectral filter at the long-wavelength end of the spectral range SB-B1 lies in a range of approximately 640 nm to approximately 740 nm, more preferably in a range of approximately 660 nm to approximately 720 nm.

In other preferred refinements, the 50% transmission value of the observation spectral filter at the long-wavelength end of the spectral range SB-B1 lies in a range of approximately 720 nm to approximately 780 nm, more preferably in a range of approximately 730 nm to approximately 760 nm.

In yet other preferred refinements, the 50% transmission value of the observation spectral filter at the long-wavelength end of the spectral range SB-B1 lies in a range of approximately 660 nm to approximately 720 nm, more preferably in a range of approximately 670 nm to approximately 710 nm.

In other preferred refinements, the 50% transmission value of the observation spectral filter at the short-wavelength end of the spectral range SB-B2 lies in a range of approximately 780 nm to approximately 850 nm, more preferably in a range of approximately 800 nm to approximately 830 nm.

In yet other preferred refinements, the 50% transmission value of the observation spectral filter at the short-wavelength end of the spectral range SB-B2 lies in a range of approximately 790 nm to approximately 840 nm, more preferably in a range of approximately 800 nm to approximately 830 nm.

In yet other preferred refinements, the 50% transmission value of the observation spectral filter at the short-wavelength end of the spectral range SB-B2 lies in a range of approximately 740 nm to approximately 810 nm, more preferably in a range of approximately 750 nm to approximately 780 nm.

The excitation spectral range SB-A, in which the illumination spectral filter is transmitting, is accordingly matched to the respectively selected pass bands of the observation spectral filter.

The transmission characteristics of the observation spectral filter arid the excitation spectral filter, in particular the aforementioned 50% transmission values, are selected depending on the considered fluorescent substance. However, the present invention is not limited to a particular fluorescent substance.

In a further preferred refinement, in the first mode of operation, an IR spectral filter is additionally arranged in the illumination path.

The advantage of this is that the heat influx into the target area as a result of the illumination light is reduced.

Accordingly, in the second mode of operation, an IR spectral filter preferably can be additionally arranged in the illumination path.

In the case of the two aforementioned measures, a light source can advantageously he used, which emits white light with a high light intensity, wherein the white light can also have significant infrared components, like the light from an incandescent lamp, short-arc lamp or the like, while the additionally provided IR spectral filters prevent excessive heat radiation from being led into the target area.

In further preferred refinements, the observation system has an endoscope or a microscope.

Herein, it is advantageous that both the light supply along the illumination path and the return of the observation light out of the target area together can be integrated into the endoscope or microscope, as a result of which there is no need for a light supply instrument that is separate from the observation instrument. The endoscope or microscope can be equipped with or without a camera.

In a further preferred refinement, the observation system has a camera, which is sensitive in the visible range and in the adjacent near infrared, and which is connected to the illumination system via a communication interface, in particular a field bus, by means of which the first or the second mode of operation is synchronized.

Herein, it is advantageous that the switching between the fluorescence mode and the white-light mode can be controlled by the user of the apparatus, for example by means of the head buttons of the camera. In this case, “synchronized” means that the operating states of camera and illumination system are automatically adapted or matched to one another. For the IR range, the camera can have a separate image recorder optimized for the IR range.

Further advantages and features emerge from the following description and the attached drawing.

It is understood that the aforementioned features and the features still to be explained below can be used not only in the respectively specified combination, but also in other combinations or on their own, without departing from the scope of the present invention.

### BRIEF DESCRIPTION OF THE DRAWINGS

Exemplary embodiments of the invention are illustrated in the drawing and described in more detail below with reference thereto, in which:

FIG. 1 shows a schematic illustration of an apparatus for fluorescence diagnosis;

FIG. 2 shows the basic principles of transmission characteristics of an observation spectral filter and an illumination spectral filter in a diagram;

FIG. 3 shows a diagram of a transmission characteristic of an exemplary embodiment of an illumination spectral filter;

FIG. 4 shows a diagram of a transmission characteristic of an exemplary embodiment of an observation spectral filter; and

FIG. 5 shows a diagram of a common illustration of the transmission characteristics of the observation spectral filter and the illumination spectral filter as per the exemplary embodiments in FIGS. 3 and 4.

### DETAILED DESCRIPTION OF PREFERRED EXEMPLARY EMBODIMENTS

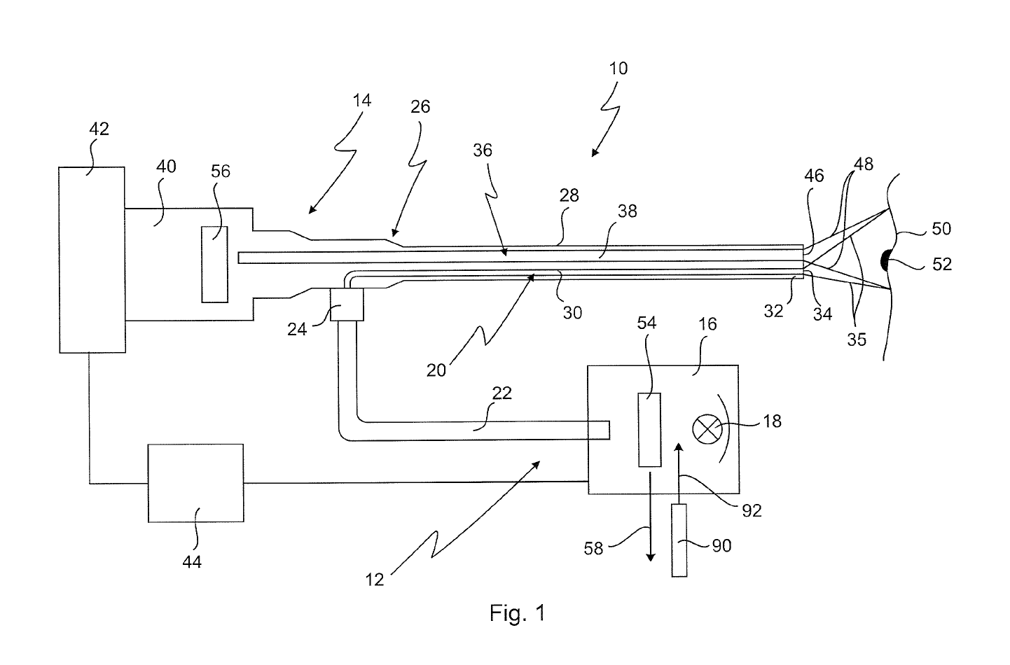

FIG. 1 schematically illustrates an apparatus for fluorescence diagnosis provided with the general reference sign 10. Without loss of generality, the apparatus 10 will be described in the following text on the basis of a use for medical fluorescence diagnosis. However, the apparatus 10 can also be used for technical fluorescence diagnosis for industrial or scientific purposes.

In general, the apparatus 10 has an illumination system 12 and an observation system 14.

The illumination system 12 has a light source 16, which is designed to generate white light. To this end, the light source 16 has a lamp 18 or a lamp system, for example a xenon discharge lamp. However, other lamps or lamp systems generating white light can also be used, such as arc discharge lamps, incandescent lamps, LED lamp systems and the like.

In terms of the present invention, “white light” should be understood to be polychromatic light, or light with a broad spectral band, which has a continuous or quasi-continuous spectrum over at least the visible spectral range.

The illumination system 12 furthermore has an illumination path 20. In the illustrated exemplary embodiment, the illumination path 20, starting from the light source 16, is in part implemented by an optical waveguide cable 22, which is connected to an optical waveguide connector 24 of an endoscope 26. The endoscope 26 has an elongate shaft 28, through which the illumination path 20 continues to extend in the form of an optical waveguide 30, for example in the form of a fibre-optic bundle, starting from the optical waveguide connector 24 to a distal end 32 of the shaft 28. If the light source 16 is switched on, the light leaves via a light-exit surface 34 according to a light-exit cone 35.

Thus, the illumination path 20 is partly integrated into the endoscope 26.

The observation system 14 has an observation path 36, which is likewise at least in part integrated into the endoscope 26. In the endoscope 26, the observation path 36 can be implemented in a number of ways, for example by a coherent fibre-optic bundle 38, by a relay lens system or, in the case where the endoscope 26 is designed as a video endoscope, by an image sensor, arranged, for example, in the region of the distal end 32, with the image transmission then being carried out by electrical signal lines, which run through the shaft 28, to a proximal end 40 of the endoscope.

The apparatus 10 can also have a microscope instead of the endoscope 26

However, in the shown exemplary embodiment, a camera 42 is connected to the proximal end 40 of the endoscope 26.

The camera 42 is connected to the illumination system 12 via a communication interface 44. The communication interface 44 can have a field bus, in particular a CAN bus with a CAN-bus protocol.

The observation system 14 can receive observation light via a light-entry surface 46 at the distal end 32 of the shaft 28 of the endoscope 26, as indicated by a light cone 48.

Starting from the light source 16, illumination light, which is emitted by the light-exit surface 34, is directed at a target area 50 via the illumination path 20. By way of example, the target area 50 is a tissue region in the human or animal body, which is intended to be examined for the presence of malignant tissue. The light coming from the target area 50 enters the observation path 36 of the observation system 14 via the light-entry surface 46 and the camera 42 detects said light.

The apparatus 10 can be operated in two operating modes, to be precise in a first operating mode in which a fluorescent substance 52 in the target area 50 is excited to fluoresce by the illumination system 12 and the fluorescence is observed by means of the observation system 14, and in a second operating mode, in which the target area 50 is illuminated by white light by means of the illumination system 12 and the thus illuminated target area 50 is observed by means of the observation system 14 like in classic endoscopy. The first operating mode will be referred to as the fluorescence mode in the following text and the second operating mode will be referred to as the white-light mode in the following text.

In the present case, the term “fluorescent substance” should be understood in general terms. The term comprises fluorescent dyes, pigments and the like, and, in principle, comprises all substances that can be excited to fluoresce. In the present case, the fluorescent substance 52 can be such a substance which only forms in the tissue by a biochemical process as a result of dispensing a precursor substance to a patient and which accumulates more strongly in certain tissue regions, or the fluorescent substance 52 can have been applied directly in the target area SO. However, the fluorescent substance 52 can also be a substance intrinsic to the body, which has thus not been dispensed to the patient, and which can be excited to autofluoresce.

So that the apparatus 10 can operate in the fluorescence mode and in the white-light mode, the illumination system 12 has an illumination spectral filter 54 and the observation system 14 has an observation spectral filter 56.

The illumination spectral filter 54 can be arranged at any point in the illumination path 20 of the illumination system 12, wherein, in the illustrated exemplary embodiment, the illumination spectral filter 54 is arranged in the light source 16. It is likewise possible for the observation spectral filter 56 to be arranged at any point in the observation path 36, with said filter in this case being arranged in the region of the proximal end 40 of the endoscope 26. In particular, the observation spectral filter 56 can also be arranged on the light-entry side of the image sensor of the camera 42. The observation spectral filter 54 can be designed as an interference filter.

In the fluorescence mode, the illumination spectral filter 54 is arranged in the illumination path 20, whereas said filter is removed from the illumination path 20 in the white-light mode, as indicated by an arrow 58. By contrast, the observation spectral filter 56 is arranged in the observation path 36 of the observation system 14 in both the fluorescence mode and the white-light mode. This is made possible by the embodiment of the observation spectral filter 56 still to be described in the following text.

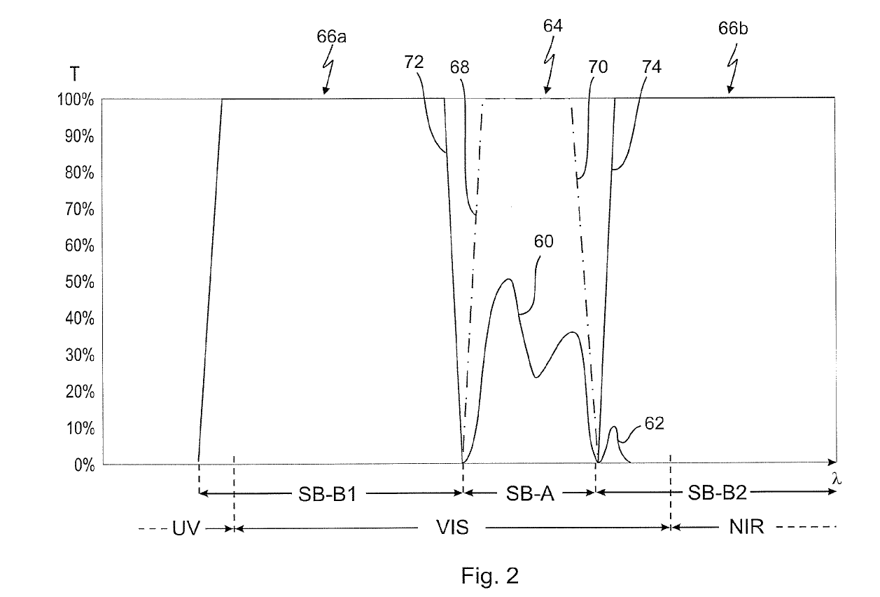

With reference to FIG. 2, the transmission characteristics of the observation spectral filter 56 and the illumination spectral filter 54 are firstly described in general.

FIG. 2 shows a diagram, not to scale, in which the wavelength λ is plotted on the abscissa and the transmission T is plotted on the ordinate.

In FIG. 2, VIS denotes the visible spectral range, the short-wavelength end of which lies at approximately λ=380 nm and the long-wavelength end of which lies at approximately λ=750 nm, The short-wavelength side of the visible spectral range VIS is adjoined by the ultraviolet spectral range UV, and the long-wavelength side of the visible spectral range VIS is adjoined by the spectral range of the near infrared NIR.

As already described above, the light source 16 generates white light, which is correspondingly composed of the continuous spectrum of the visible spectral range VIS, with it also being possible for the white light to have spectral components in the UV spectral range and in the NIR spectral range.

An absorption curve 60 in FIG. 2 illustrates the absorption spectrum of the fluorescent substance 52. An excitation spectral range SB-A of the illumination spectral filter 54 lies in a spectral range in which the fluorescent substance 52 is most absorbent or which range includes a suitable subregion of the absorption spectrum of the fluorescent substance 52, and in this case said excitation spectral range lies in the visible spectral range VIS. A curve 62 in FIG. 2 illustrates the fluorescence spectrum of the fluorescent substance 52, wherein the fluorescence spectrum lies partly outside of the excitation spectral range SB-A on the long-wavelength side. The excitation spectral range SB-A is not necessarily identical to the absorption spectral range of the fluorescent substance, but can also be wider or narrower than the absorption spectral range.

The illumination spectral filter 54 has a transmission characteristic, which is represented by a dash-dotted transmission function 64 in FIG. 2. According to the transmission function 64, the illumination spectral filter 54 is transmitting (transparent) in the excitation spectral range SB-A, i.e. it has a high transmission T in that range, while the illumination spectral filter 54 is substantially non-transmitting (opaque) on both the long-wavelength side outside of the excitation spectral range SB-A and on the short-wavelength side outside of the excitation spectral range SB-A, i.e. it has a transmission of approximately 0% in these spectral ranges, where a transmission of 0% cannot be achieved for technical reasons, however a transmission of, for example, less than 0.1% can.

The observation spectral filter 56 has a transmission characteristic, according to which the observation spectral filter 56 has two pass bands. In FIG. 2, a first pass band is represented by a transmission partial function 66a and a second pass band is represented by a transmission partial function 66b.

According to the transmission partial function 66a, the observation spectral filter 56 is transmitting (transparent) in a spectral range SB-B1 on the short-wavelength side outside of the excitation spectral range SB-A, i.e. it has a high transmission in the spectral range SB-B1. The spectral range SB-B1 extends from the short-wavelength end of the excitation spectral range SB-A up to or into the UV spectral range.

According to the transmission partial function 66b, the observation spectral filter 56 likewise has a high transmission in a spectral range SB-B2 on the long-wavelength side outside of the excitation spectral range SB-A. The spectral range SB-B2 extends from the long-wavelength end of the excitation spectral range SB-A up to or into the NIR spectral range.

In the excitation spectral range SB-A, the observation spectral filter 56 is substantially non-transmitting (opaque), i.e. the observation spectral filter 56 has a transmission of close to 0% in the excitation spectral range SB-A, wherein, as is yet to be explained below, a low remaining transmission of less than 5% of the observation spectral filter 56 in the excitation spectral range SB-A is or can be advantageous.

Thus, according to FIG. 2, the observation spectral filter 56 does not only have a high transmission on the long-wavelength side of the excitation spectral range SB-A, but also on the short-wavelength side of the excitation spectral range SB-A and there it has a high transmission over the visible spectral partial range up to the UV spectral range or into the latter. As a result of this, the observation spectral filter 56 can also remain in the observation path in the white-light mode.

To the extent that the term “high transmission” was used above, this should be understood to mean a transmission of over 50%, preferably of up to almost 100%, wherein a transmission of 100% cannot be implemented from a technical point of view, but a transmission which is very close to 100%, e.g. greater than or equal to 90% or greater than or equal to 95%, can be implemented.

It also becomes clear from FIG. 2 that the transmission characteristics of the illumination spectral filter 54 and the observation spectral filter 56, represented by the transmission functions 66a, 66b and 64, are designed to he complementary to one another.

The transmission function 64 of the illumination spectral filter 54 substantially has a trapezoidal profile, which has an edge 68 at the short-wavelength end and an edge 70 at the long-wavelength end, which edges rise steeply from the transmission minimum at approximately 0% up to the transmission maximum at approximately 100%. The gradient of the edges 68, 70 here lies in a range of approximately 100%/(45 nm) to 100%/(10 nm), wherein the steepest technically achievable edge is preferred for the transmission function of the illumination spectral filter 54.

The transmission function 66a, 66b of the observation spectral filter 56 likewise has a profile, which has an edge 72 at the short-wavelength end adjoining the edge 68 of the transmission function 64 of the illumination spectral filter 54 and an edge 74 at the end adjoining the edge 70 of the transmission function 64 of the illumination spectral filter 54, wherein the edges 72 and 74 have a gradient between the transmission minimum of approximately 0% and the transmission maximum of approximately 100% in the region of approximately 100%/(45 nm) to 100%/(10 nm). As the gradient of the edges 68, 70 and 72 and 74 increases, it is possible to select an ever-smaller spectral distance between the edges 72 and 68 and the edges 70 and 74, as a result of which the pass bands of the observation spectral filter 56 in the spectral ranges SB-B1 and SB-B2 can be brought very close to the excitation spectral range SB-A. Thus, the least amount possible of spectral components is lost in the white-light mode and so a largely natural colour image can be obtained in the white-light mode.

The illustrated linearity of the edges 68, 70, 72, 74 in this case only is a simplified illustration of the highest edge steepness possible, which is preferred for the invention.

In the fluorescence mode, the illumination spectral filter 54 is arranged in the illumination path 20. Only the spectrum in the excitation spectral range SB-A of the white light generated by the light source 16 is applied to the target area 50 via the illumination path 20. In the process, the fluorescent substance 52 is excited to fluoresce, wherein the observation spectral filter 56 passes the fluorescence spectrum as per the curve 62 in FIG. 2, while the illumination light reflected from the target area 50 is basically not transmitted by the observation spectral filter 56. However, as mentioned previously, the observation spectral filter 56 has a small remaining transmission of less than 5% in the excitation spectral range SB-A and so the observation spectral filter 56 likewise transmits a small amount of illumination light from the excitation spectral range SB-A, as a result of which the colour contrast between the fluorescence of the fluorescent substance 52 to be observed and the remaining regions in the target area 50 and the orientation in the fluorescence image are improved.

In the white-light mode, the illumination spectral filter 54 is removed from the illumination path 20 and so now the entire spectrum of the white light reaches the target area 50 via the illumination path 20, and said target area is illuminated by white light. The white light reflected in the target area 50 is transmitted by the observation spectral filter 56 except for the spectrum of the white light in the excitation spectral range SB-A and so, provided the excitation spectral range SB-A is narrow, only a small spectral range of the white light is masked, as a result of which a natural colour image display, which can still be tolerated, of the target area 50 is made possible by the camera 42. In order to improve the colour sensation, it is possible to white-balance by corresponding image processing measures.

For the transmission characteristics of the illumination spectral filter 54 and the observation spectral filter 56, there should be an optimization in respect of, on the one hand, a sufficient excitation of the fluorescent substance 52 to fluoresce and a very perceptible fluorescence display in the fluorescence mode and in respect of, on the other hand, a most natural colour display possible of the observed target area 50 in the white-light mode. In the following text, this will be described on the basis of the spectral position of the edges 70 and 74 on the one hand and the edges 68 and 72 on the other hand.

The effect of shifting the edges 70 and 74 to longer wavelengths is that the observed fluorescence intensity is reduced because the observation spectral filter 56 then only transmits a part of the fluorescence spectrum. In particular, this is critical if the fluorescence spectral range is very close to the excitation spectral range SB-A, as illustrated in FIG. 2. Conversely, the effect of a shift of the edges 70 and 74 to shorter wavelengths is that the illumination spectral filter 54 cuts out a portion of the illumination light in the excitation spectral range SB-A, which light is required to excite the fluorescence. It is for these reasons that the position of the edges 70 and 74 of the transmission functions of the illumination spectral filter 54 and the observation spectral filter 56 at the long-wavelength end of the excitation spectral range SBA should be selected as a function of the fluorescent substance 52.

By contrast, if the excitation spectral range SB-A already lies very close to the long-wavelength end of the visible spectral range VIS, a shift of the edges 70 and 74 to longer or shorter wavelengths has no or only little effect on the lifelike colour display of the target area 50 in the white-light mode. However, if the excitation spectral range SB-A lies further in the visible spectral range VIS, a shift of the edges 70 and 74 also has an effect on the colour fidelity of the target area 50, observed by the observation system 14, because shifting the edge 74 could then mask spectral components required for the lifelike colour reproduction. This is explained in more detail in the following text on the basis of the edges 68 and 72.

A shift of the edges 68 and 72 to longer wavelengths is disadvantageous in that the illumination light spectrum required for exciting the fluorescence in the excitation spectral range SB-A is reduced. This reduces the quality of the fluorescence display in the fluorescence mode. However, if the excitation spectral range SB-A lies close to the long-wavelength end of the visible spectral range VIS as illustrated in FIG. 2, a shift of the edges 68, 72 to longer wavelengths has the advantage that the observation spectral filter 56 masks fewer spectral components of the excitation spectral range SB-A, as a result of which the colour reproduction becomes more lifelike. Thus, an optimum must be found for the position of the edges 68, 72 that allows for the two aforementioned effects of shifting the edges 68, 72.

Such an optimum can be found with the aid of the L*a*b* colour space according to the standards ISO 12647 and ISO 13655. The colour deviation ΔE that can be derived therefrom constitutes a measure for how large the colour deviation of an image filtered by the observation spectral filter 56 is compared to an image in which the white light is completely detected by the observation system 14. Herein, the fact also has to be taken into account that the colour deviation, specifically in the case of body tissue, especially the mucous membrane, acts differently than for example in the case of the image of a white surface.

The optimum of the position of the edges 68, 72 should satisfy the requirement that the colour deviation ΔE is not greater than 4.0, which, although it is perceived as a colour difference by the human eye, can however still be tolerated.

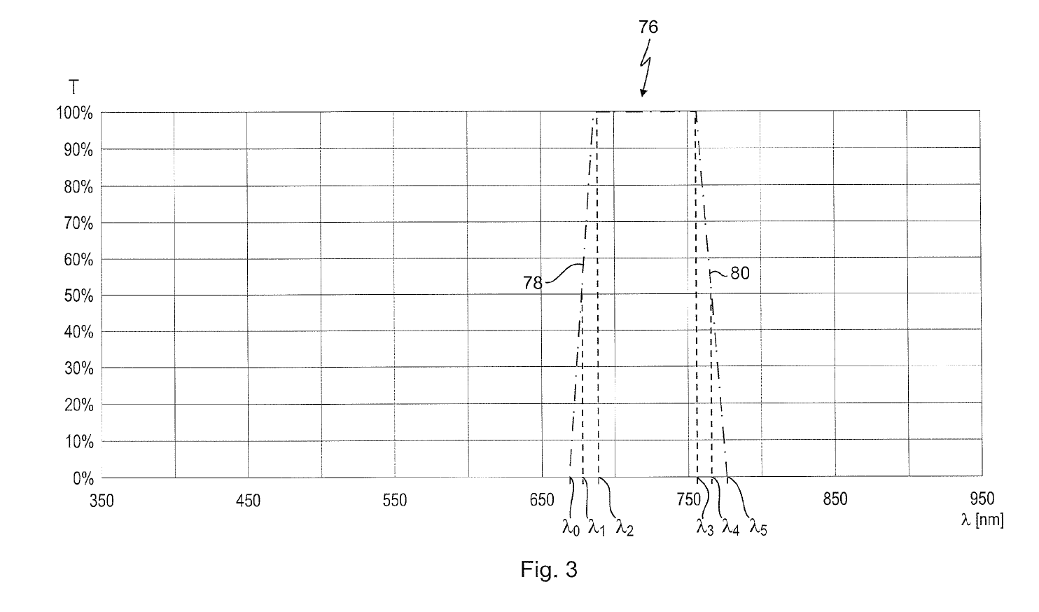

In the following text, an exemplary embodiment of transmission characteristics of the illumination spectral filter 54 and the observation spectral filter 56 is explained with reference to FIGS. 3 to 5.

FIG. 3 shows a schematic transmission function 76 of the illumination spectral filter 54. At λ₀=667.5 nm, the transmission T of the illumination spectral filter 54 is still approximately 0%, at λ₁=675 nm, the transmission T is approximately 50% and at λ₂=682.5 nm, the transmission T is approximately 100%. The increase in the transmission function 76 from the transmission minimum at λ₀ to the transmission maximum at λ₂ is steep and has a gradient of 100%/(15 nm). From λ₂ to λ₃=757.5 nm, the transmission T of the illumination spectral filter 54 remains at a maximum, it is still approximately 50% at λ₄=765 nm, and sinks to approximately 0% at λ₅=772.5 nm. Like the edge 78, the edge 80 thus has a gradient of approximately 100%/(15 nm). Here, the illustrated linearity of the edges 78, 80 is in turn only an illustrative simplification.

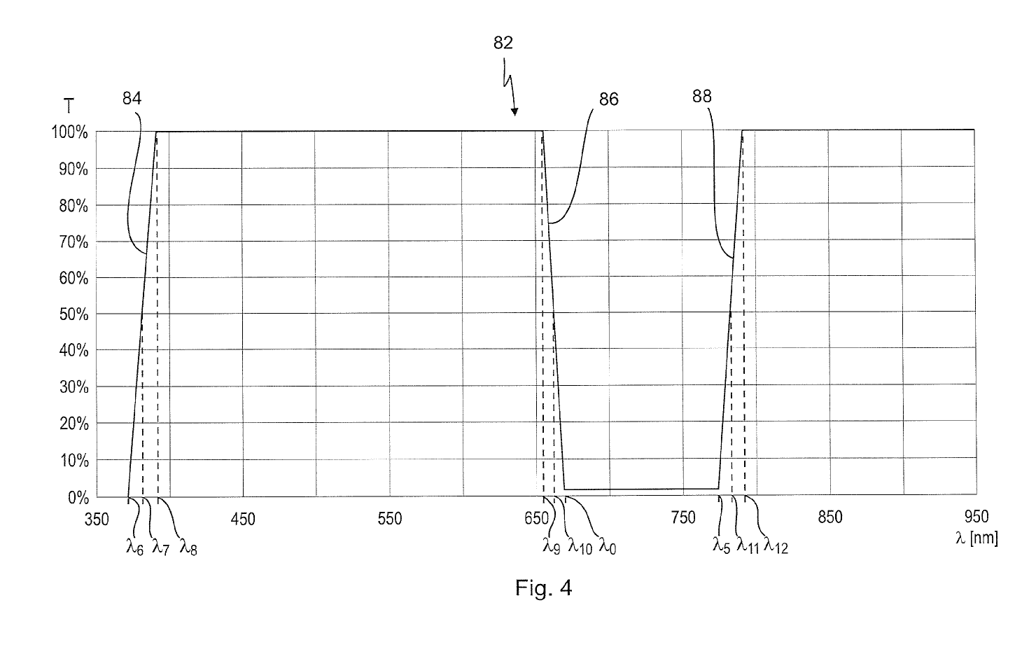

FIG. 4 shows a schematic transmission function 82 of the observation spectral filter 56. The transmission T of the observation spectral filter 56 still is approximately 0% at λ₆=375 nm, is approximately 50% at λ₇=382.5 nm and reaches approximately 100% at λ₈=390 nm. The transmission T of the observation spectral filter 56 remains at a maximum until λ₉=652.5 nm, then falls along an edge 86 to approximately 50% at λ₁₀=660 nm and to 2% at λ₀=667.5 nm. In the spectral range from λ₀=667.5 nm to λ₅=772.5 nm, i.e. in the pass band of the illumination spectral filter 54, the transmission T of the observation spectral filter 56 is approximately 2%. Along a further edge 88, the transmission rises again to approximately 50% at λ₁₁=780 nm, and again reaches the maximum value of approximately 100% at λ₁₂=787.5 nm, which in this case remains up to 950 nm.

In this example, the average gradient of the edges 84, 86 and 88 is 100%/(15 nm) in each case.

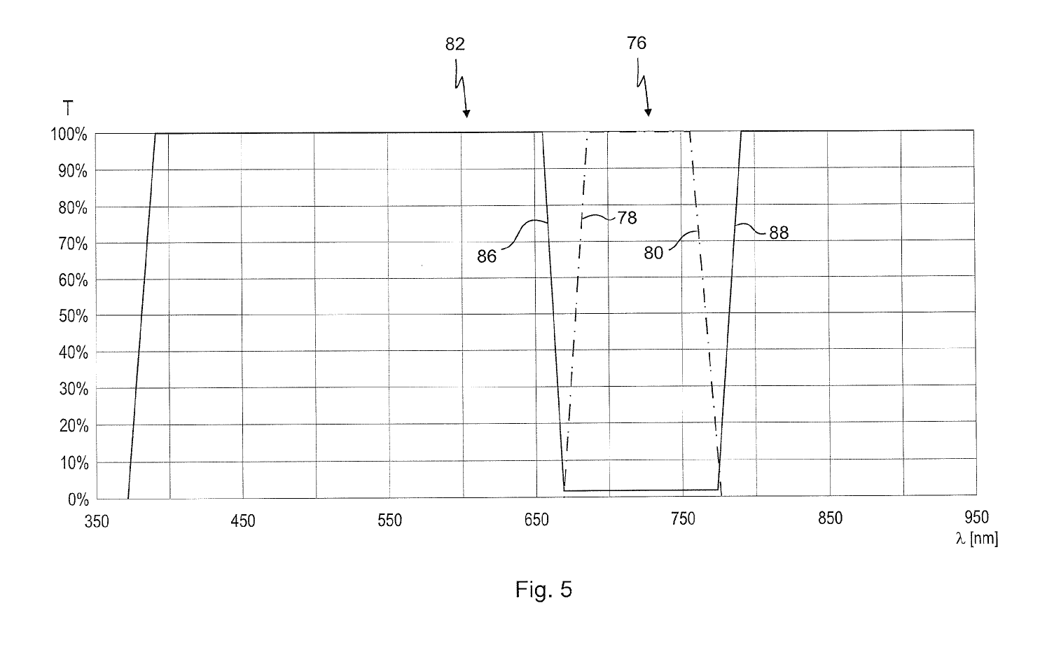

FIG. 5 shows the two transmission functions 76 and 82 in a common diagram, from which it can be gathered that the transmission functions 76, of the illumination spectral filter 54, and 82, of the observation spectral filter 56, are complementary to one another. In the spectral range from λ=667.5 nm to λ=772.5 nm, the illumination spectral filter 54 has a high transmission, and the observation spectral filter 56 has a low remaining transmission of approximately 2% and so part of the illumination light used to excite the fluorescent substance 52 is added to the fluorescence image in the fluorescence mode.

In the case of the positions of the edges 86 and 78 of the transmission functions 76 and 82, this results in a colour deviation ΔE of less than 3. In the white-light mode, the observed tissue then seems to be slightly discoloured bluish-green, but this can be tolerated. By white-balancing, the colour distortion can be further reduced.

It emerges from FIGS. 3 to 5 that the 50% transmission values of the transmission functions 76 and 82 have a spectral distance of approximately 15 nm at the short-wavelength end and the long-wavelength end of the pass hand of the illumination spectral filter 54, i.e. the pass bands of the observation spectral filter 56 lie spectrally very close to the pass band of the illumination spectral filter 54 on both sides.

In the following text, examples for the 50% transmission values of the observation spectral filter (56) at the long-wavelength end of the spectral range SB-B1 (λ₁₀) and at the short-wavelength end of the spectral range SB-B2 (λ₁₁) are specified for the dye indocyanine green (ICG) and its aggregates formed in vivo, which comprise both monomers and dimers/oligomers.

For the dye ICG overall, λ₁₀ is selected in the region of between approximately 640 nm and approximately 740 nm, preferably in the region of between approximately 660 nm and approximately 720 nm. λ₁₁ is selected in the region of between approximately 780 nm and approximately 850 nm, preferably in the region of between approximately 800 nm and approximately 830 nm.

For the monomers formed in the body from ICG, λ₁₀ is selected in the region of between approximately 720 nm and approximately 780 nm, preferably in the region of between approximately 730 nm and approximately 760 nm. λ₁₁ correspondingly is selected in the region of between approximately 790 nm and approximately 840 nm, preferably in the region of between approximately 800 nm and approximately 830 nm.

For dimers or oligomers formed in the body from ICG, λ₁₀ is selected in the region of between approximately 660 nm and approximately 720 nm, preferably in the region of between 670 nm and approximately 710 nm. λ₁₁ correspondingly is selected in the region of between approximately 740 nm and 810 nm, preferably in the region of between approximately 750 nm and approximately 780 nm.

The 50% transmission values of the illumination spectral filter 54 at the short-wavelength end (λ₁) of the excitation spectral range SB-A and at the long-wavelength end (λ₄) of the excitation spectral range SB-A are matched to the selected values for λ₁₀ and λ₁₁ in each individual case.

Referring back to FIG. 1, in the fluorescence mode, provision is furthermore made in the apparatus 10 for an IR spectral filter 90 to be able additionally to be arranged in the illumination path 20, as indicated by an arrow 92. The IR spectral filter 90 or another IR spectral filter likewise can be arranged in the illumination path 20 in the white-light mode. Such an IR filter 90 can avoid that the heat influx into the body and other parts of the system becomes too large by correspondingly blocking IR light. However, in the process, the transmission properties of the IR filter 90 have to be selected such that said filter does not disturb the white-light observation and fluorescence observation respectively in the white-light mode or in the fluorescence mode.

The fluorescence mode and the white-light mode can be synchronized between the camera and the illumination system 12 by means of the communication interface 44, which for example can be designed like a field bus and by means of which the camera 42 is connected to the illumination system 12. Here, the communication via the field bus can be wired or wireless.

What is claimed is: Patent Claims

1. An apparatus for fluorescence diagnosis, comprising:
an illumination system for illuminating a target area, said illumination system having
a light source designed for generating white light,
an illumination path,
an illumination spectral filter, said illumination spectral filter being transmitting in an excitation spectral range and substantially non-transmitting in a spectral range on a long-wavelength side outside of said excitation spectral range,
said illumination spectral filter being arranged in said illumination path in a first illumination mode and being removed from said illumination path in a second illumination mode,
an observation system for observing said target area, said observation system having
an observation path,
an observation spectral filter, said observation spectral filter being substantially non-transmitting in said excitation spectral range and transmitting in said spectral range on said long-wavelength side outside of said excitation spectral range, and further being transmitting in a spectral range on a short-wavelength side outside of said excitation spectral range,
said observation spectral filter being arranged in said observation path in said first illumination mode and in said second illumination mode.

2. The apparatus of claim 1, wherein said excitation spectral range has a spectral width which is, at a maximum, approximately as large as an absorption spectral range of a fluorescent substance in said target area.

3. The apparatus of claim 1, wherein said observation spectral filter is transmitting at least up into the near infrared spectral range on said long-wavelength side outside of said excitation spectral range.

4. The apparatus of claim 1, wherein said observation spectral filter is transmitting up into the UV spectral range on said short-wavelength side outside of said excitation spectral range.

5. The apparatus of claim 1, wherein said illumination spectral filter has a first transmission characteristic, and said observation spectral filter has a second transmission characteristic, wherein said first transmission characteristic is complementary to said second transmission characteristic.

6. The apparatus of claim 1, wherein said observation spectral filter has a transmission in a range of between approximately 0.05% and approximately 5% in said excitation spectral range.

7. The apparatus of claim 1, wherein said illumination spectral filter has a first transmission function having an edge at at least one of a short-wavelength end and a long-wavelength end of said first transmission function, said edge having a gradient between a transmission minimum and a transmission maximum in a range of approximately 100%/(45 nm) to approximately 100%/(10 nm).

8. The apparatus of claim 7, wherein said observation spectral filter has a second transmission function having an edge at at least one of a short-wavelength end and a long-wavelength end adjacent to said first transmission function, said edge having a gradient between a transmission minimum and a transmission maximum in a range of approximately 100%/(45 nm) to approximately 100%/(10 nm).

9. The apparatus of claim 1, wherein a spectral distance between a 50% transmission value of said observation spectral filter and a 50% transmission value of said illumination spectral filter at at least one of a short-wavelength and a long-wavelength end of said excitation spectral range is less than approximately 45 nm.

10. The apparatus of claim 1, wherein a spectral distance between a 50% transmission value of said observation spectral filter and a 50% transmission value of said illumination spectral filter at at least one of a short-wavelength and a long-wavelength end of said excitation spectral range is less than approximately 20 nm.

11. The apparatus of claim 1, wherein a spectral distance between a 50% transmission value of said observation spectral filter and a 50% transmission value of said illumination spectral filter at at least one of a short-wavelength and a long-wavelength end of said excitation spectral range is less than approximately 15 nm.

12. The apparatus of claim 1, wherein a 50% transmission value of said observation spectral filter at a long-wavelength end of said spectral range on said short-wavelength side outside of said excitation spectral range lies in a range of approximately 640 nm to approximately 740 nm.

13. The apparatus of claim 1, wherein a 50% transmission value of said observation spectral filter at a long-wavelength end of said spectral range on said short-wavelength side outside of said excitation spectral range lies in a range of approximately 660 nm to approximately 720 nm.

14. The apparatus of claim 1, wherein a 50% transmission value of said observation spectral filter at a short-wavelength end of said spectral range on said long-wavelength side outside of said excitation spectral range lies in a range of approximately 780 nm to approximately 850 nm.

15. The apparatus of claim 1, wherein a 50% transmission value of said observation spectral filter at a short-wavelength end of said spectral range on said long-wavelength side outside of said excitation spectral range lies in a range of approximately 800 nm to approximately 830 nm.

16. The apparatus of claim 1, wherein a 50% transmission value of said observation spectral filter at a long-wavelength end of said spectral range on said short-wavelength side outside of said excitation spectral range lies in a range of approximately 720 nm to approximately 780 nm.

17. The apparatus of claim 1, wherein a 50% transmission value of said observation spectral filter at a long-wavelength end of said spectral range on said short-wavelength side outside of said excitation spectral range lies in a range of approximately 730 nm to approximately 760 nm.

18. The apparatus of claim 1, wherein a 50% transmission value of said observation spectral filter at a short-wavelength end of said spectral range on said long-wavelength side outside of said excitation spectral range lies in a range of approximately 790 nm to approximately 840 nm.

19. The apparatus of claim 1, wherein a 50% transmission value of said observation spectral filter at a short-wavelength end of said spectral range on said long-wavelength side outside of said excitation spectral range lies in a range of approximately 800 nm to approximately 830 nm.

20. The apparatus of claim 1, wherein a 50% transmission value of said observation spectral filter at a long-wavelength end of said spectral range on said short-wavelength side outside of said excitation spectral range lies in a range of approximately 660 nm to approximately 720 nm.

21. The apparatus of claim 1, wherein a 50% transmission value of said observation spectral filter at a long-wavelength end of said spectral range on said short-wavelength side outside of said excitation spectral range lies in a range of approximately 670 nm to approximately 710 nm.

22. The apparatus of claim 1, wherein a 50% transmission value of said observation spectral filter at a short-wavelength end of said spectral range on said long-wavelength side outside of said excitation spectral range lies in a range of approximately 740 nm to approximately 810 nm.

23. The apparatus of claim 1, wherein a 50% transmission value of said observation spectral filter at a short-wavelength end of said spectral range on said long-wavelength side outside of said excitation spectral range lies in a range of approximately 750 nm to approximately 780 nm.

24. The apparatus of claim 1, wherein said observation system comprises an endoscope.

25. The apparatus of claim 1, wherein said observation system comprises a microscope.

26. The apparatus of claim 1, wherein said observation system comprises a camera, which is sensitive in the visible spectral range and in the adjacent near infrared spectral range, and which is connected to the illumination system via a communication interface, said communication interface synchronizing said first illumination mode and said second illumination mode.
